(19) United States
(12) Patent Application Publication
(10) Pub. No.: US 2021/0298669 A1
(43) Pub. Date: Sep. 30, 2021

(54) Device, System and Method for Determining Fluid Accumulation in the Lower Extremities of a Patient

(71) Applicant: BIOTRONIK SE & Co. KG, Berlin (DE)

(72) Inventor: Volker Lang, Berlin (DE)

(73) Assignee: BIOTRONIK SE & Co. KG, Berlin (DE)

(21) Appl. No.: 17/259,006

(22) PCT Filed: Jul. 22, 2019
(86) PCT No.: PCT/EP2019/069617

§ 371 Date: Jan. 8, 2021

(30) Foreign Application Priority Data
Jul. 27, 2018 (DE) 10 2018 118 228.7

(51) Int. Cl. A61B 5/00; A61B 5/0537; A61B 5/0538; A61B 5/07; G16H 50/30; G16H 40/67

(52) U.S. Cl. CPC A61B 5/4878; A61B 5/0537; A61B 5/0538; G16H 40/67; A61B 5/746; G16H 50/30; A61B 5/076

(57) ABSTRACT

The present disclosure relates to a device for determining fluid accumulation in a lower extremity of a patient. The device comprises a housing and an electrode, wherein the device is configured to measure an impedance in the lower extremity of the patient. Also, a system and a method for determining fluid accumulation in a lower extremity of a patient are disclosed.

### CROSS-REFERENCE TO RELATED APPLICATIONS

This application is the United States national phase under 35 U.S.C. § 371 of PCT International Patent Application No. PCT/EP2019/069617, filed on Jul. 22, 2019, which claims the benefit of German Patent Application No. 10 2018 118 228.7, filed on Jul. 27, 2018, the disclosures of which are hereby incorporated by reference herein in their entireties.

### TECHNICAL FIELD

The present disclosure relates to devices, systems and methods for determining fluid accumulation in the lower extremities of a patient.

### BACKGROUND

Heart failure is a severe illness and decompensation of patients with heart failure is one of the most expensive events in today's health economics since those patients need to be re-hospitalized.

Clinical practice indicates that heart failure decompensation starts with a disturbed hemostasis due to inadequate heart performance. The body is unable to remove fluid. In the early state of decompensation, the fluid is accumulated in the legs due to gravity. Later, the fluid is accumulated in the lungs. Then the patient has difficulties breathing usually resulting in hospitalization. Treatment in the hospital aims at removing the fluid by medication (e.g. with furosemide, brand name “Lasix”).

An intrathoracic impedance measurement to detect fluid accumulation in the lungs is known. Such measurement can be performed by measuring impedance between a housing of an implant (e.g. a pacemaker or an ICD, ICD—implantable cardioverter-defibrillator) and an electrode connected to the implant. If the lungs contain little fluid (e.g. water), the impedance is high. If the fluid content in the lungs rises (e.g. due to decompensation), the impedance drops to lower values. Such measurement was only partially successful as (1) the measurement vector does not measure the complete lungs thus missing fluid accumulation, and (2) fluid accumulation in the lungs happens only at a later stage of decompensation.

However, fluid removal could also be started while the patient is in an early state of decompensation. Medication (such as Lasix) can be given at home but the dosage is difficult due to side effects.

The present disclosure is directed toward overcoming one or more of the above-mentioned problems, though not necessarily limited to embodiments that do.

### SUMMARY

Based on the above, it is an object to provide improved technologies for detection of fluid accumulation in a patient. Earlier detection allows treatment of decompensation at an earlier stage leading to reduced troubles for the patient and reduced costs for the hospitals.

A device according to claim 1, a system according to claim 12, and a method according to claim 15 are provided. Further embodiments are subject matter of dependent claims.

In one aspect, a device for determining fluid accumulation (e.g. water accumulation) in a lower extremity of a patient is provided. The device comprises a housing and an electrode, and the device is configured to measure an impedance in the lower extremity of the patient.

The lower extremities of the body of a human being comprise the hip, the upper leg (thigh), the knee, the lower leg (shank), the ankle, the foot and the toes. The device may be adapted to be arranged on or in the lower leg of a patient.

The specific impedance of regular (muscle) tissue in the lower extremities and fluid (i.e. water with ion contents) is different. This difference in impedance can be used to determine fluid content in the lower extremities. The impedance measurement may be performed with extremely small current for low current consumption and below pain or feeling threshold.

The device may further comprise a battery, a transmitting module and/or an electronic module. Some or all of the before mentioned components may be arranged within the housing. The battery may be configured to provide electrical energy to all components of the device, in particular to the electronic module and to the transmitting module. The electronic module may be connected to the electrode and may be configured to determine an impedance value. For example, the impedance may be measured between the electrode and the housing (serving as counter electrode). The electrode may be arranged on the housing. The electrode may be electrically isolated from the housing. The transmitting module may be configured to transmit one or more measured impedance value(s) to a server.

The housing may comprise a biocompatible material, e.g., titanium. The housing may be made of the biocompatible material. The housing may have a length of less than 30 mm. The length of the housing may be between 10 mm and 30 mm.

The electronic module may be configured to measure impedance values in a regular interval, e.g. every 30 minutes or every hour. A timestamp may be assigned to each measured impedance value. Several measured impedance values (and their respective timestamps, if available) may be combined in a data package for transfer to the server.

In another aspect, a system for determining fluid accumulation in a lower extremity of a patient is disclosed. The system comprises a device and a server. The device comprises a housing, an electrode, and a transmitting module. The server comprises a processor and memory. The device is configured to measure an impedance in the lower extremity of the patient. The transmitting module is configured to transmit a measured impedance value to the server. The processor of the server is configured to evaluate the measured impedance value in order to determine fluid accumulation.

The system may further comprise a patient device. The patient device may act as a relay between the device and the server. Communication between the device and the patient device may be conducted via MICS (MICS—Medical Implant Communication Service, frequencies in the range from 401 MHz to 406 MHz) or Bluetooth (frequencies in the range from 2,402 GHz to 2,480 GHz, e.g. Bluetooth low energy). Communication between the patient device and the server may occur via mobile network (3G, 4G, 5G), internet, or other communication channels.

The system may further comprise a clinician device which may communicate with the server. The server may be configured to transfer measured impedance value(s) (received from the device) to the clinician device. Communication between the clinician device and the server may occur via mobile network (3G, 4G, 5G), internet, or other communication channels. The clinician device may be a personal computer (PC), a laptop, a tablet, or a smartphone.

In yet another aspect, a method for determining fluid accumulation in a lower extremity of a patient is provided. The method comprises steps of: providing a device according to the present disclosure on the lower extremity or in the lower extremity, measuring, by the device an impedance value in the lower extremity, and determining fluid accumulation in the lower extremity from the measured impedance value.

In one embodiment, the device may be an implantable medical device.

The device, in particular the implantable medical device, may comprise a flexible element which is attached to the housing, wherein the electrode is arranged at the flexible element. The flexible element may be a longitudinal flexible element, wherein its length is larger than its width and its height. The electrode may be connected with the electronic module through the flexible element (e.g. by a wire). The electrode may be arranged at a distal end of the flexible element. A proximal end of flexible element may be attached to the housing such that a longitudinal extension of the flexible element and a longitudinal extension of the housing are in the same orientation. In other words, the flexible element and the housing are arranged in line with each other. The flexible element may comprise an elastomer, e.g. silicone. The flexible element may be made of the elastomer.

A length of the flexible element may be larger than a length of the housing. The length of the flexible element and the length of the housing may have a ratio of at least 2:1, 3:1, or 4:1. With the (relatively long) flexible element, a large vector for the measurement of the impedance can be provided.

The electrode at the distal end of the flexible element and the housing (acting as counter electrode) may span a vector between 5 mm to 100 mm.

The device may comprise a further flexible element. The further flexible element may be arranged at the housing opposite to the flexible element. A further electrode may be arranged at the further flexible element, e.g. at a distal end of the further flexible element. All features of the flexible element may also apply to the further flexible element. The features disclosed for the electrode may also apply to the further electrode.

In another embodiment, the device may be an external device which is configured to be arranged on the lower extremity, wherein the housing has a first surface which is in contact with the lower extremity when the device is in a use position for measuring impedance.

A thickness of the housing may be smaller than a length of the housing. A ratio of the thickness of the housing to the length of the housing may be 1:3 or smaller (e.g. 1:4 or 1:5). The device, in particular the external device, may have a shallow housing for convenience of a patient carrying the device.

The first side of the housing may be adapted to a surface of the lower extremity. The first side of the housing may have a curved surface (adjacent the skin of the lower extremity). The first side of the housing may be concave and an opposing second side of the housing may be convex.

The device may comprise two electrodes which are arranged at diametrically opposed edges of the first side of the housing. The electrodes may be arranged at a diagonal of the first side of the housing. Hereby, the distance between the electrodes leads to a large vector for measuring the impedance between the electrodes.

The first surface of the housing may have a four-sided form. Four electrodes may be arranged at edges of the first surface. Using four electrodes improves the signal-to-noise ratio of the measured impedance values. Also, the transition between the skin and the underlying tissue can be evaluated when using four electrodes.

In all embodiments disclosed herein, the electrode(s) may be a point-shaped electrode(s). Having a small electrode surface reduces the current needed for the impedance measurement.

In the system, the processor may be further configured to compare the measured impedance value with a first threshold, and provide an alarm signal if the measured impedance value exceeds the first threshold. The alarm signal can be sent from the server to the patient device and/or to the clinician device. The alarm signal may be in form of a visible signal (e.g. a text message), or an audible signal.

The server may be further configured to store several measured impedance values in the memory hereby forming several stored impedance values. The processor may be further configured to determine a difference between the (recently received) measured impedance value and a latest stored impedance value, compare the difference with a second threshold, and provide an alarm signal if the difference exceeds the second threshold. The difference may show a decrease or an increase of the impedance.

In some embodiments, the present disclosure provides technology to (1) early detect the event of decompensation, (2) transfer the information to the treating physician, (3) allows changing the medication thus avoiding the decompensation, and (4) verifying the successful treatment.

The impedance may be measured in regular intervals (e.g. 2 per hour) over a collection period of e.g. 10 s to 60 s. Noise may be removed by averaging the measured impedance values over the collection period. Two data points per hour may be transmitted in regular intervals to the server (e.g. daily). Hourly data may be necessary to identify circadian patters as well as changes of impedance values after change of medication. Hourly data may be averaged in the server to daily data. 5-10 data points of daily data may be used to establish a base line. The daily values may be trended and compared to the baseline. If the impedance changes (e.g. >10 . . . 25%) against baseline then a “heart failure event” is being generated. This event may lead to (a) information to the patient to take a picture of the lower leg(s), (b) cross-check device signals against signal of a further device in other legs, if available, (c) information to the physician, or (d) all in a specific sequence.

In event of a characteristic impedance change, the treating physician may be alerted. The physician may then change medication (e.g. Lasix) that helps the body to remove superfluous fluid. The removal of the fluid can be monitored by the device (decreasing impedance values) as well thus helping the physician to monitor the therapy course.

An implantable device is preferable over an external device. The implantable device can be implanted in the usual position of the fluid accumulation (e.g. just above the ankles in the lower legs). Also, the skin impedance does not lower the signal-to-noise ratio.

If impedance data shows characteristic change indicative of a preceding heart failure event then the patient may be asked to take a picture of the lower legs with the smartphone. The picture of the legs together with the impedance data improves specificity of detection.

The patient may be alerted (e.g. via an app on his/her smartphone) if measured impedance values in one or both leg(s) deviate from the baseline indicating an impeding heart failure event. Based on a pre-determined treatment regime (“higher dosage”) by the physician the patient may increase the dosage of medication (e.g. Lasix). When the impedance returns to the baseline values, another pre-determined treatment regime (“normal dosage”) may be activated. The physician is able to monitor treatment regime by (a) the measured impedance values, (b) pictures of lower legs of the patient at regular intervals, and (c) by data of Lasix dosage collected by a smartphone app.

The features disclosed in regard with the system may also apply to the method and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Following, exemplary embodiments are described with reference to figures. Here show:

FIG. 1 shows a schematic representation of a device for determining fluid accumulation in a lower extremity;

FIG. 2 shows a schematic representation of a system including the device of FIG. 1, a patient device and a server;

FIG. 3 shows a schematic representation of an implantable device for determining fluid accumulation in a lower extremity;

FIG. 4 shows a schematic representation of another embodiment of an implantable device for determining fluid accumulation in a lower extremity;

FIG. 5 shows a schematic representation of an implantation of the device according to FIG. 3 or FIG. 4 in a lower leg;

FIG. 6 shows a schematic representation of another implantation of the device according to FIG. 3 or FIG. 4 in a lower leg;

FIG. 7 shows a schematic representation of a cross section of a lower leg with an implantable device according to FIG. 3 or FIG. 4;

FIG. 8 shows a schematic representation of yet another embodiment of an implantable device for determining fluid accumulation in a lower extremity;

FIG. 9 shows a schematic representation of an external device for determining fluid accumulation in a lower extremity arranged at a lower leg;

FIG. 10 shows a schematic representation of an embodiment of the external device according to FIG. 9; and

FIG. 11 shows a schematic representation of another embodiment of the external device according to FIG. 9.

### DETAILED DESCIPTION

Same reference numerals are used for same components.

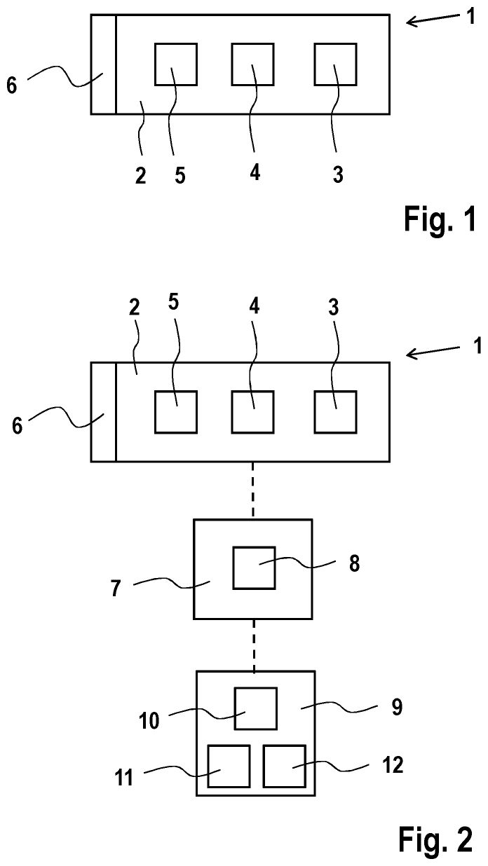

FIG. 1 shows a schematic representation of a device 1 for determining fluid accumulation in a lower extremity. The device 1 comprises a housing 2. Several components are arranged within the housing 2, namely a battery 3, a communication module 4, and an electronic module 5. An electrode 6 is arranged at the housing 2. The electrode 6 is electrically isolated with respect to the housing 2.

The battery 3 provides electrical energy to the other components including the communication module 4 and the electronic module 5. The communication module 4 is configured for wireless communication with other devices.

The electronic module 5 is connected to the electrode 6. The electronic module 5 is configured to measure an impedance between the electrode 6 and the housing 2 (acting as counter electrode).

FIG. 2 shows a schematic representation of a system including the device 1, a patient device 7 and a server 9. The patient device 7 comprises a communication unit 8. The patient device 9 acts as a relay between the device 1 and the server 9. The server 9 comprises a server communication unit 10, a processor 11 (or several processors) and memory 12 (e.g. volatile and/or non-volatile memory) for storing data.

The device 1 communicates wirelessly with the patient device 7, e.g. via Bluetooth or MICS. The measured impedance value(s) are sent from the device 1 to the patient device 7. The patient device 7 transmits the measured impedance value(s) to the server 9, e.g. via mobile radio communication. The measured impedance value(s) are evaluated by the processor 11 of the server 9 in order to determine fluid (e.g. water) accumulation in the lower extremity.

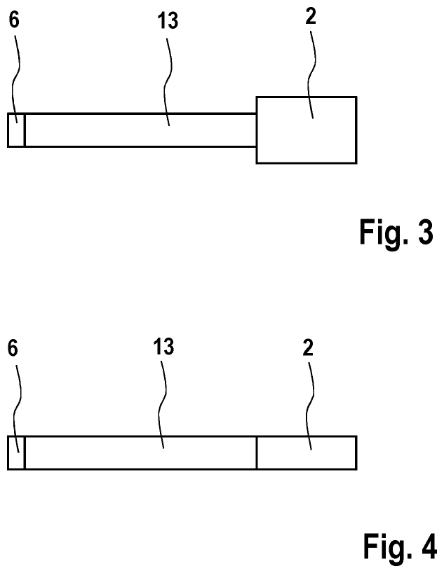

is FIG. 3 shows a schematic representation of an implantable device for determining fluid accumulation in a lower extremity. The implantable device comprises a housing 2 which may comprise the components described above with regard to FIG. 1. The implantable device further comprises a flexible (or elastic) element 13. The electrode 6 is arranged at a first end of the flexible element 13. A second end of the flexible element 13 is attached to the housing 2.

Another (but similar) embodiment of an implantable device is shown in FIG. 4. Here, the flexible element 13 and the housing 2 have the same width.

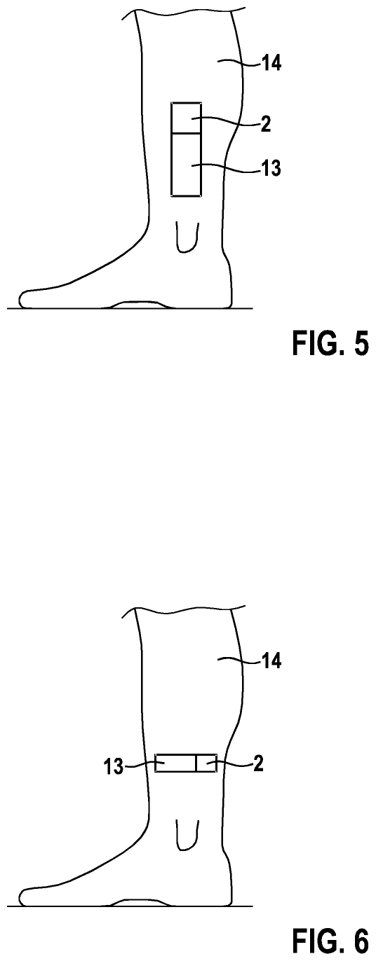

In FIG. 5, implantation of the implantable device of FIG. 3 or FIG. 4 in the lower leg 14 is shown. The electrode of the implantable device is not shown for the sake of clarity. The implantable device is implanted along a longitudinal axis of the lower leg 14.

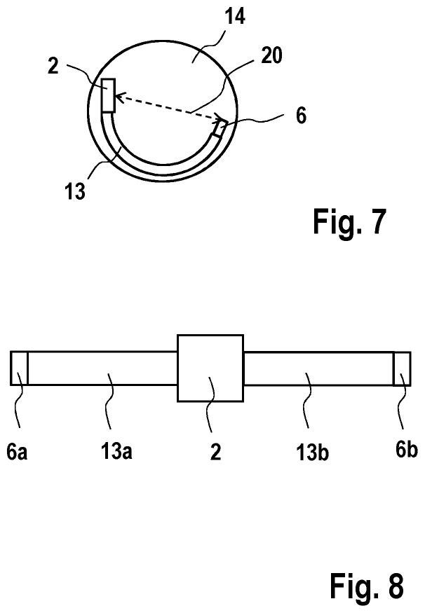

Another implantation of the implantable device is schematically shown in FIG. 6. Here, the implantable device is implanted along the circumference of the lower leg 14. The flexible element 13 bends to adapt to the lower leg 14. FIG. 7 shows a cross section of the lower leg 14 in order to illustrate the arrangement of the implantable device. Here, the housing 2 and the electrode 6 span a measuring vector 20 approximately across the width of the lower leg 14.

Another embodiment of an implantable device is shown in FIG. 8. Here, a first flexible member 13a and a second flexible member 13b extend from two opposing ends of the housing 2. A first electrode 6a is arranged at the first flexible member 13a and a second electrode 6b is arranged at the second flexible member 13b. The impedance is measured between the two electrodes 6a, 6b.

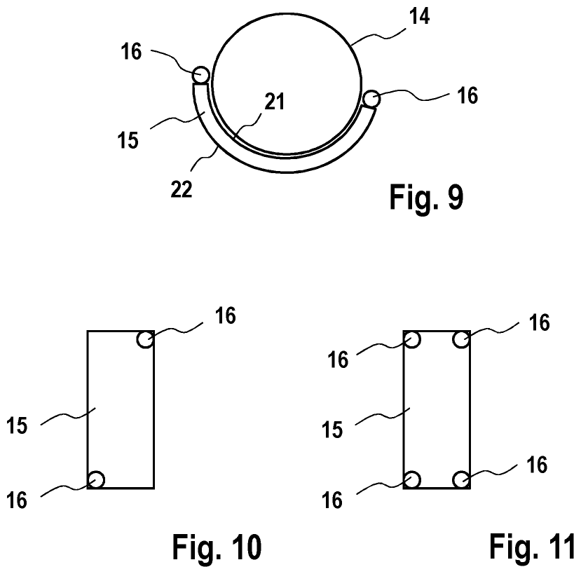

An embodiment of an external device having a housing 15 and electrodes 16 is shown in FIG. 9 (cross section of lower leg). The external device is arranged at the skin of the lower leg 14. A first surface 21 of the housing 15 is concave and adapted to be arranged on the skin of the lower leg. A second surface 22 of the housing 15 is convex. The height of the housing 15 is much smaller than its length in order to comply with patient comfort. The electrodes 16 are arranged at opposing ends of the housing 15.

Two configurations for the electrodes are shown in FIGS. 10 and 11. In FIG. 10, two electrodes 16 are arranged at diagonal edges of the housing 15 at the first surface 21 (facing the skin). In the embodiment of FIG. 11, four electrodes 16 are arranged at the four edges of the first surface 21 of the housing 15.

The solutions according to the present disclosure may have the following advantages:
Early detection of a heart failure decompensation event as fluid (water) accumulation starts in lower extremities due to gravity.
The detection is more sensitive as the measuring vector can be within the area of fluid accumulation.
Furthermore, additional “sensor” data, e.g. pictures from the lower legs, may increase the specificity even further.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCES

1 device
2 housing
3 battery
4 communication module
5 electronic module
6 electrode
7 patient device
8 communication unit
9 server
10 server communication unit
11 processor
12 memory
13 flexible element
14 lower leg
15 housing
16 electrode(s)
20 measuring vector
21 first (concave) surface of housing
22 second (convex) surface of housing

1. A device for determining fluid accumulation in a lower extremity of a patient, the device comprising a housing and an electrode, wherein the device configured to measure an impedance in the lower extremity of the patient.

2. The device of claim 1, wherein the device is an implantable medical device.

3. The device of claim 2, further comprising a flexible element which is attached to the housing, wherein the electrode is arranged at the flexible element.

4. The device of claim 3, wherein a length of the flexible element is larger than a length of the housing.

5. The device of claim 4, wherein the length of the flexible element and the length of the housing have a ratio of at least 2:1.

6. The device of claim 1, wherein the device is an external device which is configured to be arranged on the lower extremity, wherein the housing has a first surface which is in contact with the lower extremity when the device is in a use position for measuring impedance.

7. The device of claim 6, wherein a thickness of the housing is smaller than a length of the housing.

8. The device of claim 6, wherein the first surface of the housing is adapted to a surface of the lower extremity.

9. The device of claim 8, wherein the first surface of the housing is concave and an opposing second surface of the housing is convex.

10. The device of claim 6, wherein two electrodes are arranged at diametrically opposed edges of the first surface of the housing.

11. The device of claim 6, wherein the first surface has a four-sided form and four electrodes are arranged at edges of the first surface.

12. A system for determining fluid accumulation in a lower extremity of a patient, the system comprising:
a device comprising a housing, an electrode, and a transmitting module, and
a server comprising a processor and a memory,
wherein the device is configured to measure an impedance in the lower extremity of the patient,
wherein the transmitting module is configured to transmit a measured impedance value to the server, and
wherein the processor of the server is configured to evaluate the measured impedance value in order to determine fluid accumulation.

13. The system of claim 12, wherein the processor is further configured to
compare the measured impedance value with a first threshold, and
provide an alarm signal if the measured impedance value exceeds the first threshold.

14. The system of claim 12, wherein the server is further configured to store several earlier measured impedance values in the memory hereby forming several stored impedance values, wherein the processor is further configured to
determine a difference between the measured impedance value and a latest stored impedance value,
compare the difference with a second threshold, and
provide an alarm signal if the difference exceeds the second threshold.

15. A method for determining fluid accumulation in a lower extremity of a patient, comprising steps of:
providing a device according to claim 1 on the lower extremity or in the lower extremity,
measuring, by the device, an impedance value in the lower extremity, and
determining fluid accumulation in the lower extremity from the measured impedance value.
